(19) United States
(12) Patent Application Publication
(10) Pub. No.: US 2002/0169455 A1
(43) Pub. Date: Nov. 14, 2002

(54) System and method for fixation of cranial flaps

(76) Inventors: Brett Bannerman, Canyon Country, CA (US); Bruce Don Henniges, Kalamazoo, MI (US); Timothy Paul Freybler, Battle Creek, MI (US); James Keith Rains, Kalamazoo, MI (US); Edward James Mayadag, Sacramento, CA (US); Donell Gregory Diamond, Kalamazoo, MI (US)

Correspondence Address: HOWARD & HOWARD ATTORNEYS, P.C., The Pinehurst Office Center, Suite # 101, 39400 Woodward Avenue, Bloomfield Hills, MI, 48304-5151, US

(21) Appl. No.: 10/073,741

(22) Filed: Feb. 11, 2002

Related U.S. Application Data
(60) Provisional application No. 60/271,203, filed on Feb. 23, 2001.

(51) Int. Cl. A61B 17/58

(52) U.S. Cl. 606/99

(57) ABSTRACT

A system and method for fixating uses a cranial disk clamp. The cranial disk clamp includes a lower disk, an upper disk and a stem. The lower disk has an inner surface and an outer surface. The stem has first and second ends and an engaging portion between the first and second end. The stem is connected to the lower disk at the first end and extends away from the inner surface of the lower disk. The upper disk has a clamping element for releasably engaging the stem.

### CROSS-REFERENCE TO RELATED APPLICATIONS

This application claims the benefit of U.S. Provisional Patent Application Serial No. 60/271,203, Attorney Docket No. 60,500-001, filed Feb. 23, 2001 by Timothy Freybler and Brett Bannerman and entitled “Cranial Flap Fixation Device”.

### FIELD OF THE INVENTION

The present invention relates generally to a cranial bone clamp, and more specifically, to apparatus and method for fixating a cranial bone flap in an aperture of a cranium.

### BACKGROUND OF THE INVENTION

In most neurosurgical and cranial operations, it is necessary to open a large access hole or craniotomy in the skull by removing a segment of the skull or cranial bone flap. The cranial bone flap is then removed entirely or folded out of the way.

The cranial bone flap is typically formed by first drilling several holes through the skull. The holes are then connected by osteotomy cuts using a saw. The position, number, and size of the holes drilled through the skull, as well as the number of connecting osteotomies, is determined by the size, location and geometrical form of the desired bone flap and corresponding access hole. The bone flap can then be removed, exposing the underlying dura mater to provide access to the brain for the further steps of the operation.

At the end of the procedure, the previously removed bone flap or flaps are repositioned into their original locations, or in different desired locations, relative to the surrounding bone portions. This is typically accomplished in the prior art by drilling small pairs of holes in the surrounding skull bone in several places around the edge of the bone flap. Wire is then carefully threaded through the holes, taking care not to tear the dural tissue covering the brain, then twisted together to secure the edges, the ends tucked into the cut opening so that they do not puncture the skin, and the skin then stitched into place over the skull flap. The procedure is complex and time consuming, and there always is the possibility of injuring the dura either by using the high speed drills that are necessary to form the small holes or by the sharp points of the wire engaging the dura.

Another method of securing the cranial bone flap uses cranial flap tube clamps, such as those available from Synthes® Maxillofacial. These tube clamps consist of a lower clamp disk and an implant tube extending from the lower clamp disk. A top clamp disk has an aperture which slides over the implant tube. In practice, the lower clamp disk is placed beneath the skull bone at the edge of the craniotomy. The bone flap is then put back into place. The implant tube is then inserted into the aperture of the upper disk and the upper disk is lowered to the cranial surface.

In order to secure the clamp, the implant tube is inserted into a crimping device ratchet. Pressure must be exerted downward while the crimping device ratchet is actuated. Thus, the crimping device ratchet crimps the implant tube above the upper disk (thereby maintaining its position relative to the lower disk) and shears the extending portion of the implant tube.

There are several disadvantages to the Synthes Maxillofacial device. First, the crimping ratchet device is pressed downward while it is being actuated increasing the risk of damage to the dura mater and brain. Second, the crimping ratchet device leaves a portion of the implant tube extending past the upper disk.

Lastly, in order to remove the cranial flap tube clamps, a tool must inserted between the skull bone and the upper disk in order to pry the upper disk from the implant tube. Thus again, increasing the risk of damage to the dura mater and brain.

The present invention is aimed at one or more of the problems identified above.

### SUMMARY OF THE INVENTION AND ADVANTAGES

In one aspect of the present invention, a cranial disk clamp is provided. The cranial disk clamp includes a lower disk, an upper disk and a stem. The lower disk has an inner surface and an outer surface. The stem has first and second ends and an engaging portion between the first and second end. The stem is connected to the lower disk at the first end and extends away from the inner surface of the lower disk. The upper disk has a clamping element for releasably engaging the stem.

In another aspect of the present invention, a method for fixating a cranial bone flap in a craniotomy of a cranium, is provided. The method includes the steps of positioning at least one disk clamps having a lower disk relative to the cranium, placing the cranial bone flap within the craniotomy, and loosely closing the at least one disk clamps. The method further includes the step of tightening the at least one disk clamps using an application forceps tool to maintain the cranial bone flap in position.

### BRIEF DESCRIPTION OF THE DRAWINGS

Other advantages of the present invention will be readily appreciated as the same becomes better understood by reference to the following detailed description when considered in connection with the accompanying drawings wherein:

FIG. 1A is a diagrammatical illustration of a cranial disk clamp, according to an embodiment of the present invention;

FIG. 1B is a second diagrammatical illustration of the cranial disk clamp of FIG. 1A;

FIG. 2A is a top view of a lower disk of the cranial disk clamp of FIGS. 1A and 1B;

FIG. 2B is a side view of the lower disk of FIG. 2A;

FIG. 3A is a diagrammatic illustration of a stem of the cranial disk clamp of FIGS. 1A and 1B;

FIG. 3B is a side view of the stem of FIG. 3A;

FIG. 4A is a top view of an upper disk of the cranial disk clamp of FIGS. 1A and 1B;

FIG. 4B is a side view of the upper disk of FIG. 4A;

FIG. 5A is a diagrammatic illustration of a u-shaped frame of the cranial disk clamp of FIGS. 1A and 1B;

FIG. 5B is a side view of the u-shaped frame of FIG. 5A;

FIG. 6A is a diagrammatic illustration of a spring element of the cranial disk clamp of FIGS. 1A and 1B;

FIG. 6B is a side view of the spring element of FIG. 6A;

FIG. 7A is a first illustration of the use of the cranial disk clamp of FIGS. 1A and 1B;

FIG. 7B is a second illustration of the use of the cranial disk clamp of FIGS. 1A and 1B;

FIG. 7C is a third illustration of the use of the cranial disk clamp of FIGS. 1A and 1B;

FIG. 8 is a illustration of the use of an application forceps tool, according to an embodiment of the present invention;

FIG. 9A is a second illustration of the use of the application forceps tool of FIG. 8;

FIG. 9B is an illustration of the use of a cutter tool, according to an embodiment of the present invention;

FIG. 9C is a illustration of the use of a removal tool, according to an embodiment of the present invention;

FIG. 10A is a diagrammatic illustration of the application forceps tool of FIG. 8, according to an embodiment of the present invention;

FIG. 10B is a second illustration of the application forceps tool of FIG. 8;

FIG. 11A is a diagrammatic illustration of the removal tool of FIG. 9C, according to an embodiment of the present invention; and,

FIG. 11B is a second illustration of the removal tool of FIG. 9C.

### DETAILED DESCRIPTION OF THE PREFERRED EMBODIMENT

With reference to the drawings and in operation, the present invention provides a system and method for the fixation of the cranial flaps. The term cranial flap refers to a portion of the skull bone which has been cut from the skull. The cranial flap may be either entirely removed from the skull or remain partially attached to the skull.

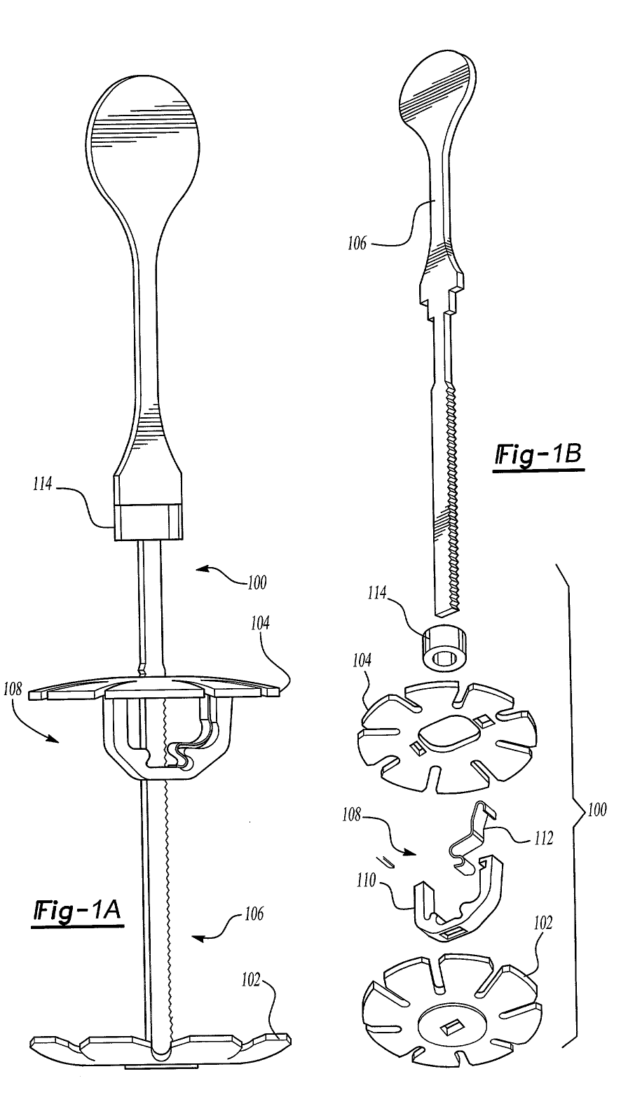

With reference to FIGS. 1A-6B, the system and method use a cranial disk clamp 1100. With specific reference to FIGS. 1A and 1B, the cranial disk clamp 100 includes a lower disk 102, an upper disk 104 and a stem 106. The cranial disk clamp 100 also includes a clamping element 108 for releasably engaging the stem 106 (see below). In one embodiment, the cranial disk clamp 100 is composed of a material suitable for implanting, such as, titanium.

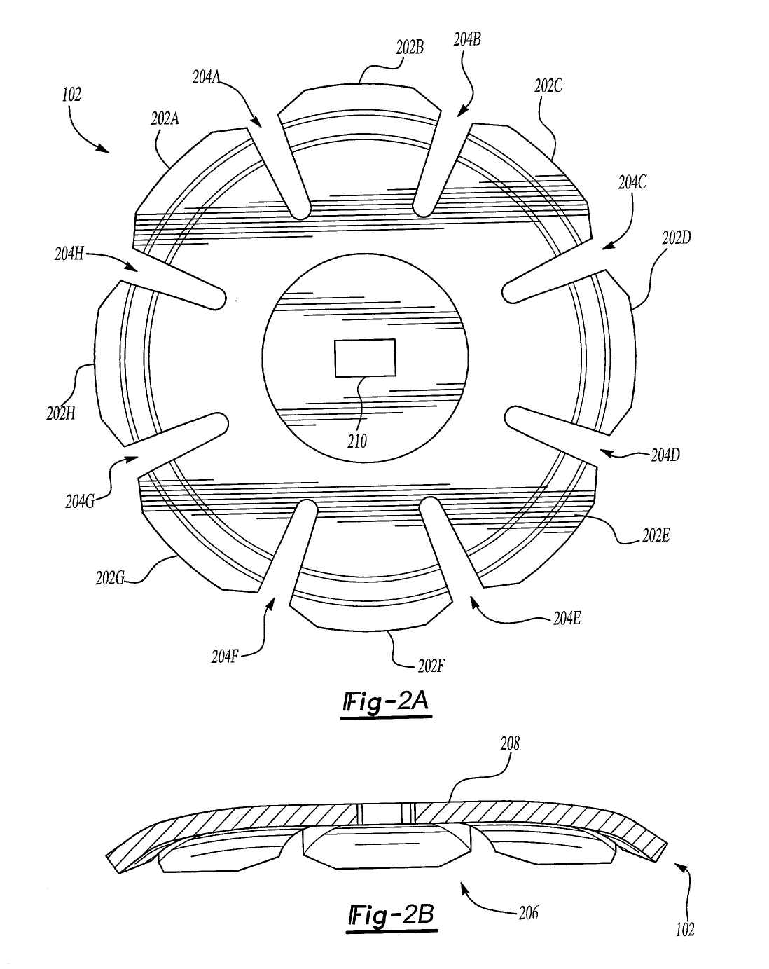

With specific reference to FIGS. 2A and 2B, the lower disk 102 has a generally circular outer shape and is curved inward toward the upper disk 104.

Preferably, the lower disk 102 has a plurality of extensions 202A-202H extending in an outward direction. The extensions 202A-202H are separated by slots 204A-204H. The embodiment shown in the Figs., has eight extensions 202A-202H and eight slots 204A-204H. It should be understood, however, that the present invention is not limited to a specific number of extensions and slots. The lower disk 102 also has an inner surface 206, an outer surface 208, and an aperture 210.

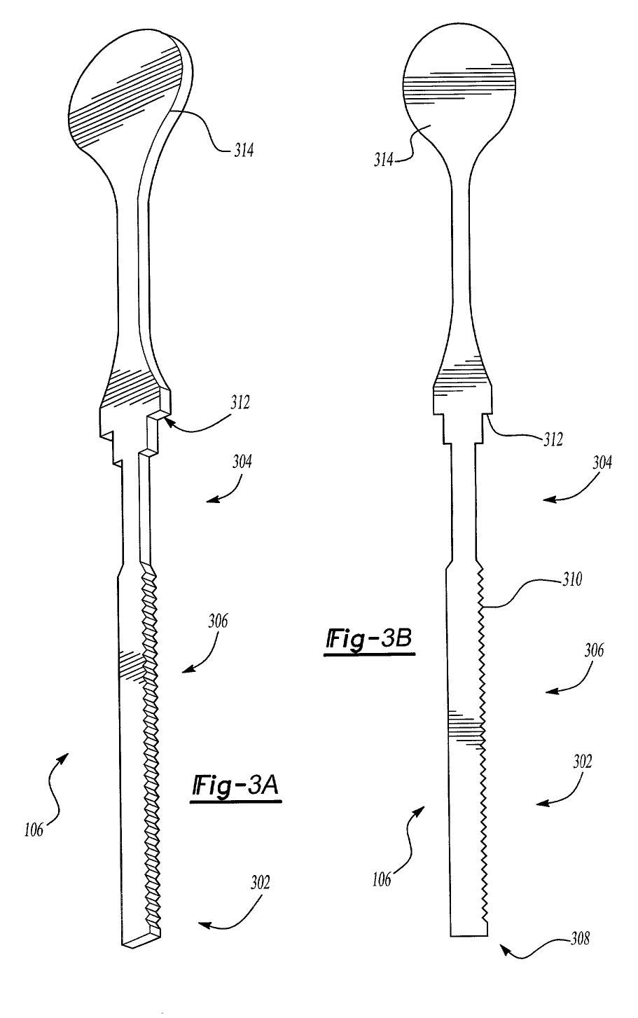

With reference to FIGS. 3A and 3B, in one embodiment, the stem 106 has a first end 302 and a second end 304. As shown in FIG. 1A, the lower disk 102 is coupled to the first end 302 of the stem 106. The stem 106 extends away from the inner surface 206 of the lower disk 102. The first end 302 of the stem 106 is located within the aperture 210 of the lower disk 102. The lower disk 102 may be fixed to the stem 106 by any suitable means, such as welding.

The stem 106 also includes an engaging portion 306 which is located between the first and second ends 302, 304. In one embodiment, the engaging portion 306 includes a plurality of teeth 308 located along an edge 310 of the stem 106. The stem 106 also includes a ridge 312 located near the second end 304 and a gripping portion 314 extending away from the second end 304 of the stem 106. The gripping portion 314 of the stem 106 is adapted to be removed by a post cutter tool (see below) after the cranial disk clamp 100 is assembled.

As shown, in the illustrated embodiment the stem 106 includes first and second sides 314A, 314B. The first and second sides 314A, 314B are larger at the gripping portion 314 than at the second end 304 of the stem 106.

In another embodiment, the stem has a circular cross-section and the gripping portion has a number of marks or engravings at the gripping portion 314.

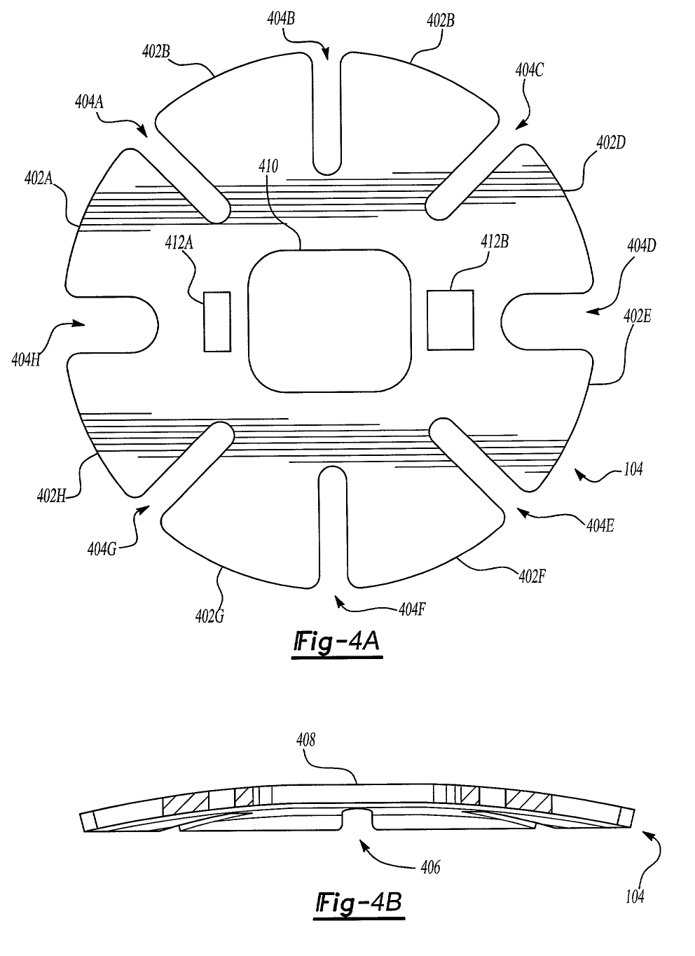

With specific reference to FIGS. 4A and 4B, the upper disk 104 has a generally circular outer shape and is curved inward toward the lower disk 102.

Preferably, the upper disk 104 has a plurality of extensions 402A-402H extending in an outward direction. The extensions 402A-402H are separated by slots 404A-404H. The embodiment shown in the Figs., the upper disk 104 has eight extensions 402A-402H and eight slots 404A-404H. It should be understood, however, that the present invention is not limited to a specific number of extensions and slots. The upper disk 104 also has an inner surface 406, an outer surface 408, and an aperture or slot 410. The upper disk 104 may also include two smaller apertures 412A, 412B on opposite sides of the apertures 410.

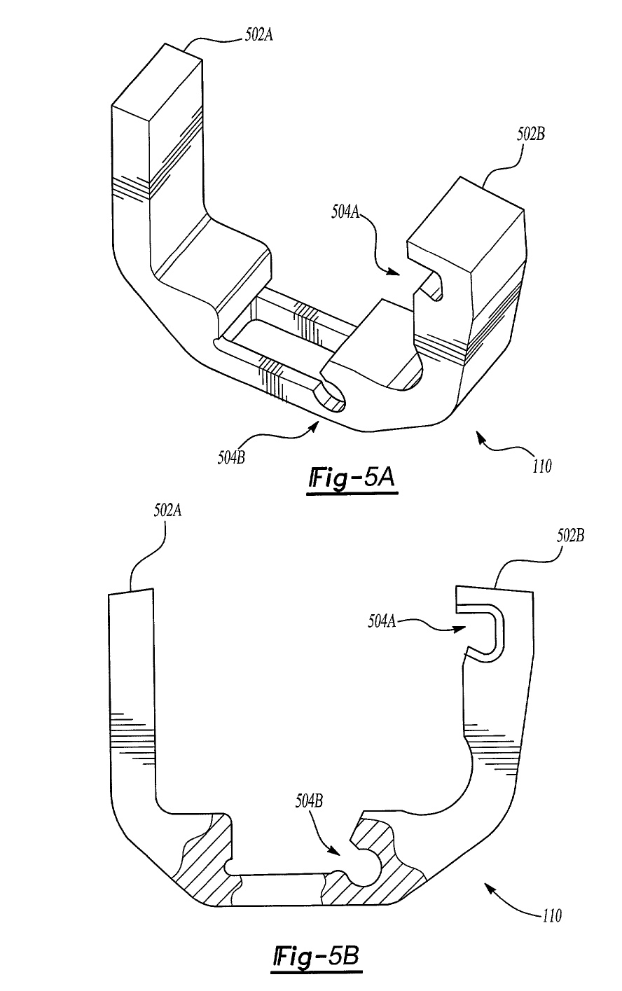

Returning to FIG. 1B, the clamping element 108 includes a generally u-shaped frame 110 and a spring element 112 coupled to the frame 110. With specific reference to FIGS. 5A and 5B, the u-shaped frame 110 includes a pair of top ends 502A, 502B. The pair of top ends 502A, 502B are connected to the inner surface 406 of the upper disk 104. In one embodiment, the pair of top ends 502A, 502B are inserted into the two smaller apertures 412A, 412B. The u-shaped frame 110 is secured to the upper disk 104 by any suitable means, such as welding.

Returning to FIGS. 5A and 5B, the u-shaped frame 110 also includes first and second receptacles 504A, 504B.

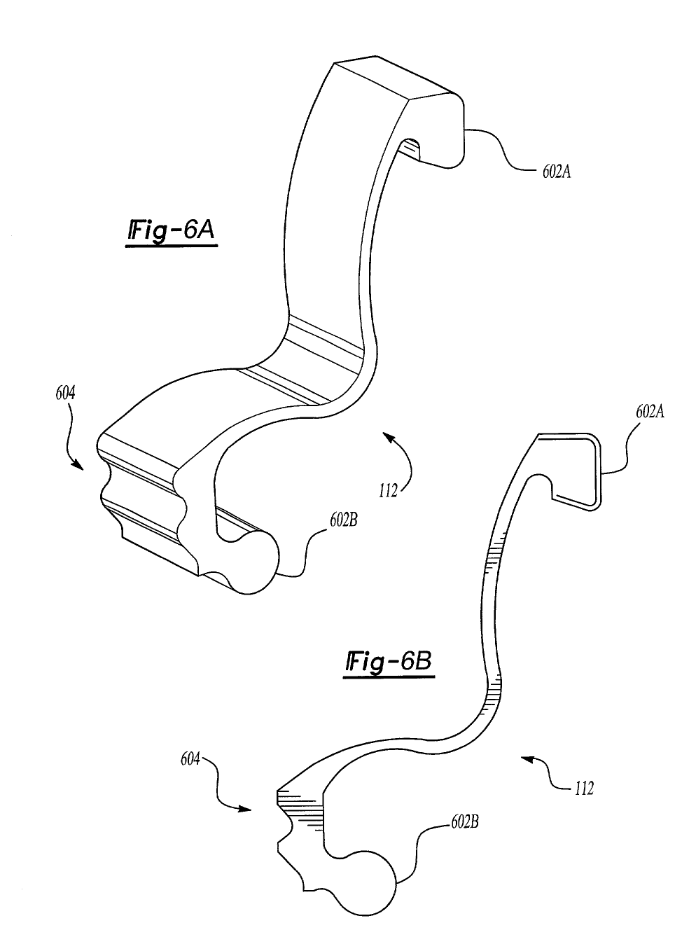

With specific reference to FIGS. 6A and 6B, spring element 112 includes first and second ends 602A, 602B. In one embodiment, the first and second ends 602A, 602B are received and held in the first and second receptacles 504A, 504B, respectively. The spring element 112 also includes an engaging surface 604 for releasably engaging the engaging portion 306 of the stem 106. In one embodiment, the engaging surface 604 and the engaging portion 306 are adapted to allow the upper disk 104 to be moved toward the lower disk 102 with minimal effort, but to restrict movement of the upper disk 104 in the opposite direction.

Returning to FIG. 4A, the aperture 410 in the upper disk 104 allows the insertion of a removal tool (see below). The removal tool is adapted to engage the spring element 112 and release the spring element 112 from engagement, i.e., an engagement position, with the engaging portion 306 of the stem 106. Thus, the upper disk 104 is allowed to move freely relative to the lower disk 102 and be removed (see below).

Returning to FIG. 1A, the cranial disk clamp 100 includes a collar 114 coupled to the stem 106 at the second end 304. In one embodiment, the collar 114 rests against the ridge 312 of the stem 106.

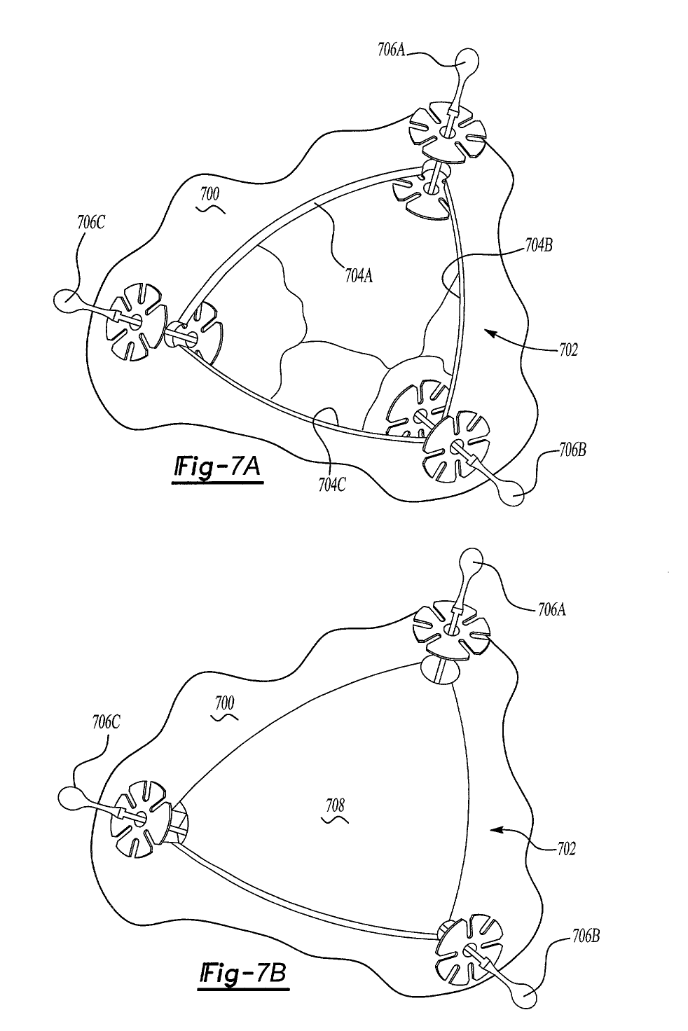

With reference to FIGS. 7A-7C, 8, and 9A-9C, use of the cranial disk clamp 100 after a neurosurgical operation will now be discussed. As shown in FIG. 7A, the neurosurgical operation was performed through a craniotomy 700 in a skull bone 702. The craniotomy 700 is shown as having a generally triangular shape. However, it should be noted that the craniotomy 700 may have any shape including rectangular, square, or circular.

The craniotomy 700 in the illustrated embodiment, has first, second and third sides 704A, 704B, 704C. After the operation has been performed, at least one cranial disk clamp 100 is placed within the craniotomy 700. If more than one disk clamp 100 is used, then the disk clamps are preferably placed at equidistant points around the craniotomy 700. In the illustrated embodiment, first, second, and third disk clamps 706A, 706B, 706C are placed at the vertices formed by the sides 704A, 704B, 704C. The lower disk 102 of each cranial disk clamp 706A, 706B, 706C is inserted underneath the skull bone 702.

With specific reference to FIG. 7B, after the cranial disk clamps are put into position, the cranial flap 708 is put back into place.

With specific reference to FIG. 7C, after the cranial flap 708 is put back into place, the upper disk 104 of each cranial disk clamp 706A, 706B, 706C is lowered by a user 710. The user 710 grips the gripping portion 314 of the stem 106 and moves the upper disk 104 towards the skull bone 702 (as shown by arrow 712).

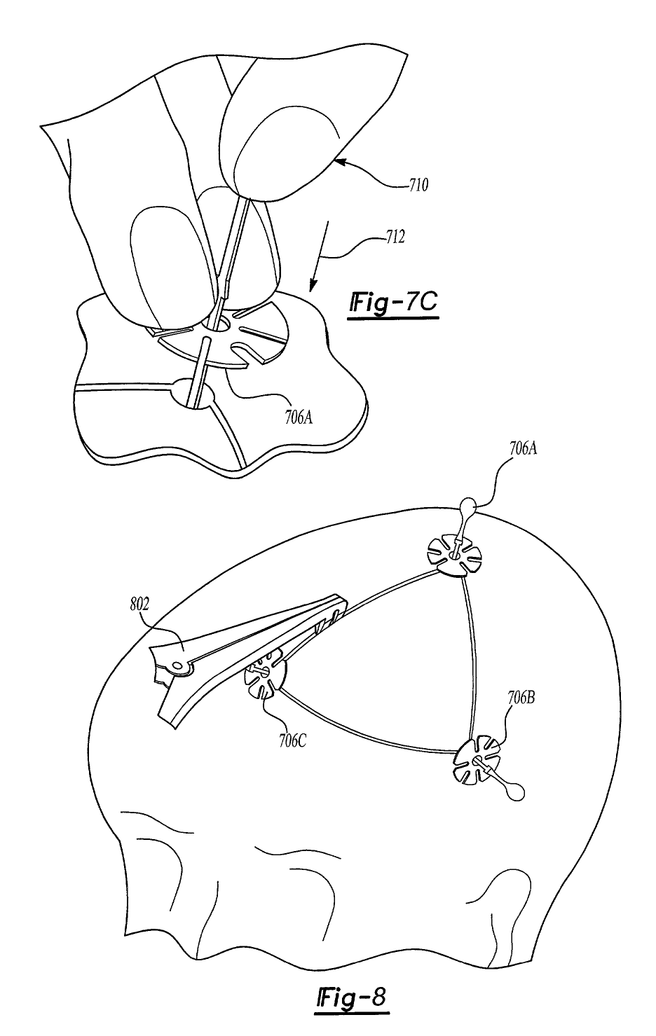

With reference to FIG. 8, after the upper disks 104 of each cranial disk clamp 706A, 706B, 706C is lowered, the position of the cranial bone flap 708 may be adjusted using an application forceps tool 802.

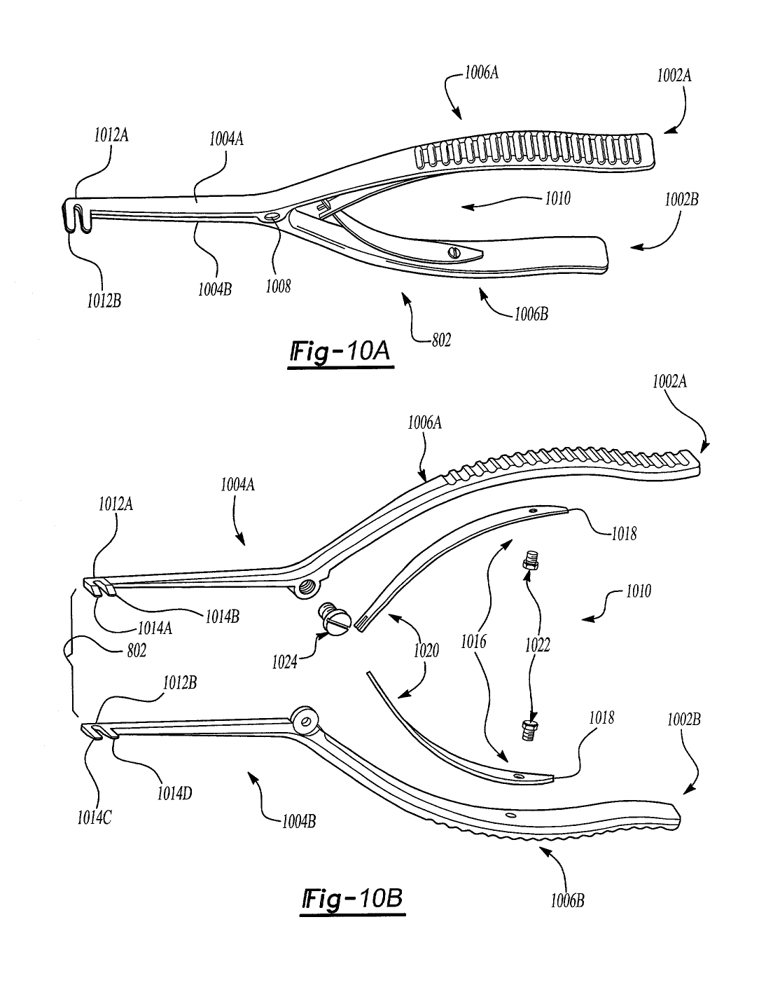

With reference to FIGS. 10A and 10B, the application forceps tool 802 includes a first handle portion 1002A and a second handle portion 1002B. The first handle portion 1002A includes a first nose end 1004A and a first gripping end 1006A. The second handle portion 1002B includes a second nose end 1004B and a second gripping end 1006B. The first and second handle portions 1002A, 1002B are pivotally coupled at a midpoint 1008 and joined by a screw 1024 at the midpoint 1008. The first and second handle portions 1002A, 1002B are movable between a first position (or normally closed position shown in FIG. 10A) and a second position (or fully open position). A spring 1010 is coupled between the first and second handle portions 1002A, 1002B. The spring 1010 biases the first and second handle portions 1002A, 1002B towards the first position.

As shown, the first handle portion 1002A and a second handle portion 1002B include ridges on an outside surface to aid in gripping the application forceps tool 802.

The application forceps tool 802 also includes a first cranial disk clamp engaging element 1012A and a second cranial disk clamp engaging element 1012B. The first and second cranial disk clamp engaging elements 1012A, 1012B are coupled to the first and second nose ends 1004A, 1004B, respectively. The first and second cranial disk clamp engaging elements 1012A,1012B is adapted to engage the collar 114 of the stem 106 and/or the upper disk 104 (see below). The first and second cranial disk clamp engaging elements 1012A, 1012B are adapted to move the upper disk 104 along the stem 106 towards the lower disk 102 when the first and second handle portions 1002A, 1002B are moved towards the second position.

As shown, the first cranial disk engaging element 1012A includes first and second prongs 1014A, 1014B which extend in a direction perpendicular from the first nose end 1004A. The second cranial disk engaging element 1012B includes third and fourth prongs 1014C, 1014D which extend in a direction perpendicular from the second nose end 1004B.

Preferably, the application forceps tool 802 is composed of a durable material, such as, stainless steel.

In one embodiment, the spring 1010 includes first and second spring elements 1016A, 1016B. Each spring element includes a first end 1018, and a second end 1020. The first ends 1018 of the first and second spring elements 1016A, 1016B are coupled to the first and second handle portions 1002A, 1002B using a screw 1022. The second ends 1020 of the first and second spring elements 1016A,1016B are connected together using a series of prongs as shown.

Additionally, at least one or both of the first and second gripping ends 1006A, 1006B are adapted to flex when a predetermined torque is applied thereto. This provides the user both a tactile and visual indication of the pressure being exerted by the application forceps tool 802.

In another embodiment, the application forceps tool 802 is adapted to limit the amount of force applied to the cranial disk clamp 100. One of the first and second handle portions 1002A, 1002B, has first and second hinged portions, one of which is adapted to slip relative to the other when a predetermined amount of force is applied thereto.

Returning to FIG. 8, with the application forceps tool 802 in the first or closed position, the first and second cranial disk engaging elements 1012A, 1012B are inserted between one of the edges 704A, 704B, 704C of the craniotomy 700 and the adjacent edge of the cranial flap 708. When the application forceps tool 802 is actuated, force is asserted against the adjacent edge of the cranial flap 708 and the opposite edge or edges of the cranial flap 708 are moved into a contacting or near contacting relationship with the respective side edge 704A, 704B, 704C of the craniotomy 700. This promotes faster healing at those edges.

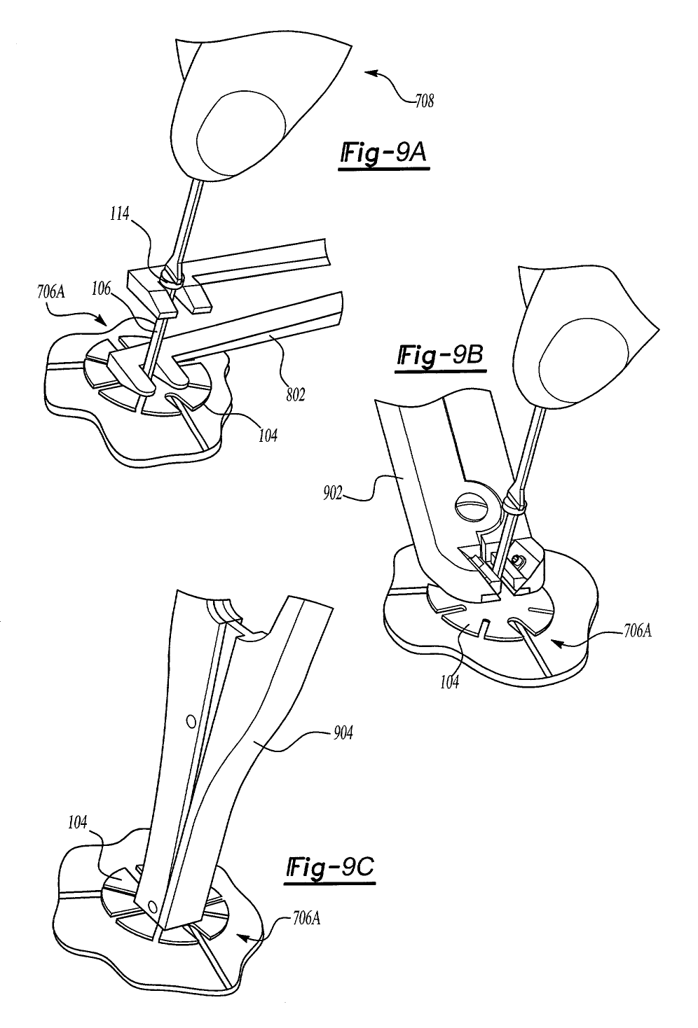

With reference to FIG. 9A, after the cranial flap 708 is moved into position, the cranial disk clamps 706A, 706B, 706C are fully tightened or engaged using the application forceps tool 802. As shown, the first and second cranial disk engaging elements 1012A, 1012B are placed around the stem 106 and the application forceps tool 802 is then opened. One of the cranial disk engaging elements 1012A, 1012B engages the collar 114 and the other engages the upper disk 104. By applying force to the application forceps tool 802, the upper disk 104 is forced towards the lower disk 102, thereby clamping the cranial flap 708 in place. In this manner, virtually no downward force is applied to the lower disk 102 and therefore, no pressure is applied to the dura matter. All of the cranial disk clamps 706A, 706B, 706C are tightened in this manner.

With reference to FIG. 9B, after all of the cranial disk clamps 706A, 706B, 706C have been tightened, the portion of the stem 106 and the gripping portion 314 extending above the upper disk 104 is removed using a cutting tool 902.

With reference to FIG. 9C, the upper disk 104 of a cranial disk clamp 706A can be easily removed using a removal tool 904.

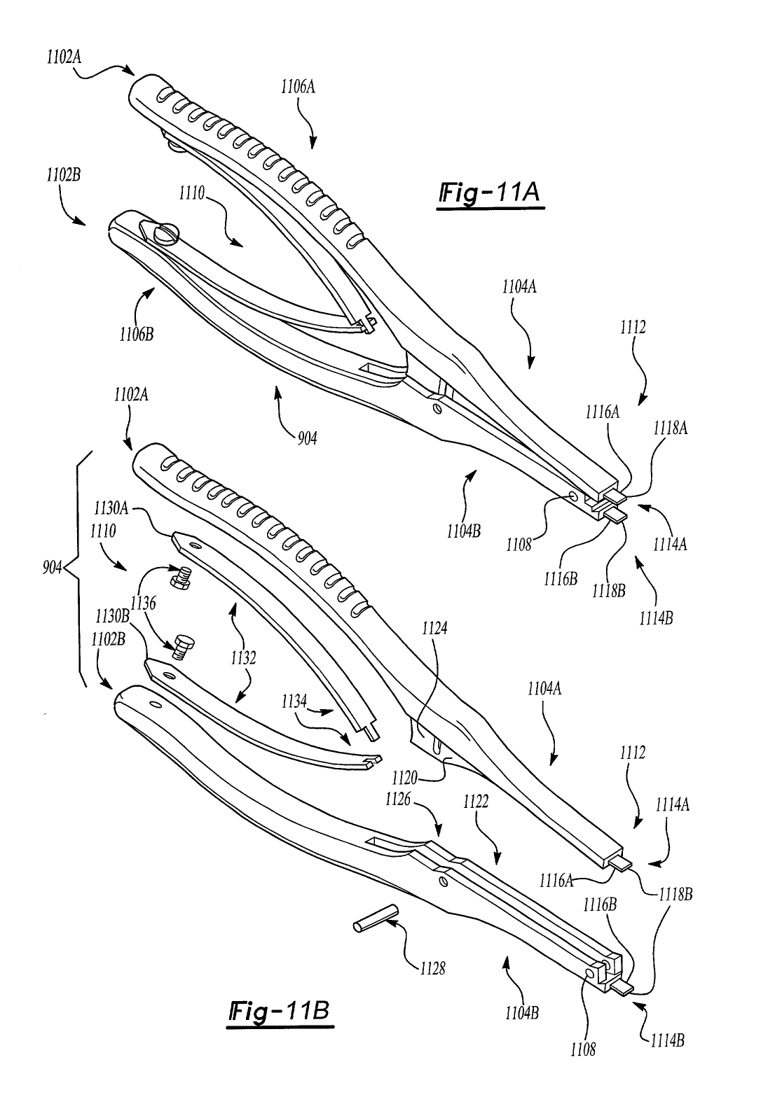

With reference to FIGS. 11A and 11B, the removal tool 904 includes first and second handle portions 1102A, 1102B. The first handle portion 1102A includes a first nose end 1104A and a first gripping end 1106A. The second handle includes a second nose end 1104B and a second gripping end 1106B. As shown, the first and second handle portions 1102A, 1102B include ridges on an outside surface to aid in gripping the removal tool 904. The first and second handle portions 1102A, 1102B are pivotally coupled at a pivot point 1108 located on the first and second nose ends 1104A, 1104B and are movable between a first position (or normally closed position) and a second position.

A spring 1110 is coupled between the first and second handle portions 1102A, 1102B. The spring 1110 biases the first and second handle portions 1102A, 1102B towards the first position.

The removal tool 904 includes a cranial disk clamp disengaging element 1112 coupled to the first and second nose ends 1104A, 1104B. The cranial disk disengaging element 1112 is adapted to be inserted into the aperture 410 of the upper disk 104 when the first and second handle portions 1102A, 1102B are in the first position. The cranial disk disengaging element 1112 is also adapted to disengage the clamping element 108 from the stem 106 when the first and second handle portions 1102A, 1102B are moved towards the second position.

In one embodiment, the cranial disk clamp disengaging element 1112 includes a first disengaging element 1114A coupled to the first nose end 1104A of the first handle portion 1102A and a second disengaging element 1114B coupled to the second nose end 1104B of the second handle portion 1102B. Preferably, the first and second disengaging elements 1114A, 1114B include a straight portion 1116A, 1116B and a tapered portion 1118A, 1118B.

With specific reference to FIG. 11B, the first handle portion 1102A includes an alignment insert 1120 which extends along an interior surface of the first nose end 1104A. The second handle portion 1102B includes an alignment slot 1122 which extends along an interior surface of the second nose end 1104B. The alignment insert 1120 and the alignment slot 1122 having an aligning relationship, i.e., maintain the first and second handle portions 1102A, 1102B in alignment. In the embodiment illustrated, the alignment insert 1120 includes an inner aperture 1124 and the alignment slot 1122 includes a pair of outer apertures 1126. A pivot pin 1128 is inserted through the inner and outer apertures 1124, 1126.

Preferably the removal tool 904 is composed of a durable material, such as stainless steel.

In one embodiment, the spring 1110 includes first and second spring elements 1130A, 1130B. Each spring element 1130A, 1130B includes a first end 1132 and a second end 1134. The first ends 1132 of the first and second spring elements 1130A, 1130B are coupled to the first and second handle portions 1102A, 1102B using a screw 1136. The second ends 1134 of the first and second spring elements 1130A, 1130B are connected together using a series of prongs as shown.

The removal tool 904 is used by inserting the tip of the removal tool 904 in the aperture 410 of the upper disk 104. The removal tool 904 is actuated by movement of the first and second handle portions 1102A,1102B towards the second position to disengage the clamping element 108 by moving the spring element 112 away from the stem 106. The removal tool 904 is then moved in a direction away from the stem 106 to remove the upper disk 104.

Obviously, many modifications and variations of the present invention are possible in light of the above teachings. The invention may be practiced otherwise than as specifically described within the scope of the appended claims.

What is claimed is:

1. A cranial disk clamp, comprising:
a lower disk having an inner surface and an outer surface;
a stem having first and second ends, and an engaging portion between the first and second ends and being connected to the lower disk at the first end and extending away from the inner surface of the lower disk; and,
an upper disk having a clamping element for releasably engaging the stem.

2. A cranial disk clamp, as set forth in claim 1, wherein the lower disk and the upper disk are composed of a material suitable for implanting.

3. A cranial disk clamp, as set forth in claim 1, wherein the lower disk has a generally circular outer shape and is curved inward toward upper disk.

4. A cranial disk clamp, as set forth in claim 3, wherein the lower disk has a plurality of extensions extending in an outward direction separated by slots.

5. A cranial disk clamp, as set forth in claim 1, wherein the upper disk has a generally circular outer shape and is curved inward toward the lower disk.

6. A cranial disk clamp, as set forth in claim 5, wherein the upper disk has a plurality of extensions extending in an outward direction separated by slots.

7. A cranial disk clamp, as set forth in claim 1, wherein the engaging portion of the stem is composed of a plurality of teeth along an edge of the stem.

8. A cranial disk clamp, as set forth in claim 1, further comprising a gripping portion extending away from the second end of the stem.

9. A cranial disk clamp, as set forth in claim 1, wherein the stem has first and second sides, the first and second sides being larger at the gripping portion than at the second end of the stem.

10. A cranial disk clamp, as set forth in claim 1, wherein the upper disk includes an inner surface and an outer surface and the clamping element includes a generally u-shaped frame and a spring element, the u-shaped frame having a pair of top ends connected to the inner surface of the upper disk, the spring element being coupled to the u-shaped frame.

11. A cranial disk clamp, as set forth in claim 10, wherein the spring element includes first and second ends coupled to the u-shaped frame and an engaging surface for releasably engaging the engaging portion of the stem.

12. A cranial disk clamp, as set forth in claim 10, wherein the upper disk includes an aperture adapted to allow insertion of a removal tool, the spring element being adapted to be released from an engaging position by the removal tool.

13. A cranial disk clamp, as set forth in claim 1, wherein the stem includes a gripping portion extending away from the second end of the stem, the gripping portion of the stem being adapted to be removed by a post cutter tool after the cranial disk clamp is assembled.

14. A cranial disk clamp, comprising:
a lower disk having an inner surface and an outer surface;
a stem having first and second ends, a gripping portion extending from the second end, and an engaging portion between the first and second ends and being connected to the lower disk at the first end and extending away from the inner surface of the lower disk;
a collar coupled to the stem at the second end; and,
an upper disk having an inner surface, an outer surface, an aperture and a clamping element, the stem being positioned through the aperture and the upper disk being moveable between a first position and a second position along the stem, the clamping element being adapted to allow movement of the upper disk in a direction towards the first position and to releasably restrict movement of the upper disk in a direction towards the second position.

15. A cranial disk clamp, as set forth in claim 14 wherein the lower disk and the upper disk are composed of a material suitable for implanting.

16. A cranial disk clamp, as set forth in claim 14, wherein the lower disk has a generally circular outer shape and is curved inward toward the second end of the stem.

17. A cranial disk clamp, as set forth in claim 16, wherein the lower disk has a plurality of extensions extending in an outward direction separated by slots.

18. A cranial disk clamp, as set forth in claim 14, wherein the upper disk has a generally circular outer shape and is curved inward toward the first end of the stem.

19. A cranial disk clamp, as set forth in claim 18, wherein the upper disk has a plurality of extensions extending in an outward direction separated by slots.

20. A cranial disk clamp, as set forth in claim 14, wherein the upper disk includes an inner surface and an outer surface an d the clamping element includes a generally u-shaped frame and a spring element, the u-shaped frame having a pair of top ends connected to the inner surface of the upper disk, the spring element being coupled to the u-shaped frame.

21. A cranial disk clamp, as set forth in claim 20, wherein the spring element includes first and second ends coupled to the u-shaped frame and an engaging surface for releasably engaging the engaging portion of the stem.

22. A cranial disk clamp, as set forth in claim 20, wherein the upper disk includes an aperture adapted to allow the insertion of a removal tool, the spring element being adapted to be released from an engaging position by the removal tool.

23. A cranial disk clamp, as set forth in claim 14, wherein the gripping portion of the stem is adapted to be removed by a post cutter tool after the cranial disk clamp is assembled.

24. A removal tool for a cranial disk clamp, the cranial disk clamp having a lower disk and an upper disk, the upper disk having an aperture and a clamping element for releasably engaging the lower disk, comprising:
a first handle portion having a first nose end and a first gripping end;
a second handle portion having a second nose end and a second gripping end, the first and second handle portions being pivotally coupled at the first and second nose ends and being movable between a first position and a second position;
a spring coupled between the first and second handle portions for biasing the first and second handle portions towards the first position;
a cranial disk clamp disengaging element coupled to the first and second nose ends and being adapted to be inserted into the aperture of the upper disk when the first and second handle portions are in the first position and to disengage the clamping element from the lower disk when the first and second handle portions are moved towards the second position.

25. A removal tool, as set forth in claim 24, wherein the first handle portion includes an alignment insert extending along an interior surface of the first nose end and the second handle portion includes an alignment slot extending along an interior surface of the second nose end, the alignment insert and the alignment slot having an aligning relationship.

26. A removal tool, as set forth in claim 25, wherein the alignment insert includes an inner aperture and the alignment slot includes a pair of outer apertures and further comprising a pivot pin inserted through the inner and outer apertures.

27. A removal tool, as set forth in claim 24, wherein the first and second handle portions are composed of a durable material.

28. A removal tool, as set forth in claim 24, wherein the cranial disk clamp disengaging element is composed of a durable material.

29. A removal tool, as set forth in claim 24, wherein the cranial disk clamp disengaging element includes a first disengaging element coupled to the first nose end of the first handle portion and a second disengaging element coupled to the second nose end of the second handle portion.

30. A removal tool, as set forth in claim 29, wherein the first and second disengaging elements includes a straight portion and a tapered portion.

31. A removal tool, as set forth in claim 24, wherein the spring includes first and second spring elements.

32. A removal tool, as set forth in claim 31, wherein the first and second spring elements have a first end and a second end, the first ends of the first and second spring elements being coupled to the first and second handle portions, respectively, and the second ends of the first and second spring element being connected together.

33. An application forceps tool for a cranial disk clamp, the cranial disk clamp having a lower disk, an upper disk, and a stem, the stem extending from the lower disk and having a collar and, the upper disk having an aperture and a clamping element for releasably engaging the stem, comprising:
a first handle portion having a first nose end and a first gripping end;
a second handle portion having a second nose end and a second gripping end, the first and second handle portions being pivotally coupled at a midpoint, the first and second handle portions being movable between a first position and a second position;
a spring coupled between the first and second handle portions for biasing the first and second handle portions towards the first position;
a first cranial disk clamp engaging element coupled to the first nose end and being adapted to engage the collar of the stem;
a second cranial disk clamp engaging element coupled to the second nose end and being adapted to engage the upper disk, wherein the first and second cranial disk clamp engaging elements are adapted to move the upper disk along the stem towards the lower disk when the first and second handle portions are moved towards the second position.

34. An application forceps tool, as set forth in claim 33, wherein the first and second cranial disk clamp engaging elements include first and second prongs extending in a direction perpendicular from the first and second nose ends, respectively.

35. An application forceps tool, as set forth in claim 33, wherein the first and second handle portions are composed of a durable material.

36. An application forceps tool, as set forth in claim 33, wherein the cranial disk clamp engaging elements are composed of a durable material.

37. An application forceps tool, as set forth in claim 33, wherein the spring includes first and second spring elements.

38. An application forceps tool, as set forth in claim 37, wherein the first and second spring elements have a first end and a second end, the first ends of the first and second spring elements being coupled to the first and second handle portions, respectively, and the second ends of the first and second spring element being connected together.

39. An application forceps tool, as set forth in claim 33, wherein the application forceps tool is adapted to limit the force applied to the cranial disk clamp.

40. An application forceps tool, as set forth in claim 33, wherein at least one of the first and second gripping ends are adapted to flex when a predetermined torque is applied thereto.

41. A method for fixating a cranial bone flap in a craniotomy of a cranium; including the steps of:
positioning at least one disk clamp having a lower disk relative to the cranium;
placing the cranial bone flap within the craniotomy;
loosely closing the at least one disk clamp;
positioning the cranial bone flap using an application forceps tool to allow for bone to bone contact on at least one side of the cranial bone flap; and,
tightening the at least one disk clamps to maintain the cranial bone flap in position.

42. A method for fixating a cranial bone flap in a craniotomy of a cranium; including the steps of:
positioning at least one disk clamp having a lower disk relative to the cranium;
placing the cranial bone flap within the craniotomy; and,
loosely closing the at least one disk clamp by a user without tools.

43. A method, as set forth in claim 42, including the step of tightening the at least one disk clamp using an application forceps tool to maintain the cranial bone flap in position.

44. A method, as set forth in claim 42, wherein the cranial disk clamp includes a lower disk, an upper disk, and a stem, the stem having a collar and extending from the lower disk, the upper disk having an aperture and a clamping element for releasably engaging the stem, and wherein the step of tightening the method includes the steps of:
positioning the application forceps tool between the collar and the upper disk; and
actuating the application forceps tool to force the upper disk away from the collar.

45. A method, as set forth in claim 44, including the step of using a cutting tool to remove a portion of the stem extending past the upper disk.

46. A method, as set forth in claim 45, wherein the portion of the stem removed extends below an upper contour of the upper disk such that a remaining portion of the stem does not extend above the upper contour.

47. A method for removing a disk clamp positioned within a cranium, including the steps of:
inserting a tip of a removal tool in a center slot of an upper disk of the disk clamp;
actuating the removal tool while pushing the removal tool towards the disk clamp to disengage the disk clamp; and,
moving the removal tool in a direction away from the disk clamp to remove the upper disk of the disk clamp.
